(19) United States
(12) Patent Application Publication
(10) Pub. No.: US 2008/0243152 A1
(43) Pub. Date: Oct. 2, 2008

(54) Atraumatic Surgical Band

(75) Inventors: Pascal Paganon, Serezin du Rhone (FR); Jean-Paul Gilbert Ricol, Saint Germaine sur l'Abresle (FR)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: COMPAGNIE EUROPEENNE D'ETUDE ET DE RECHERCHE DE DISPOSITIFS POUR I'IMPLANTATION PAR LAPAROSCOPIE

(21) Appl. No.: 10/598,101

(22) PCT Filed: Feb. 18, 2005
(86) PCT No.: PCT/FR2005/000387

§ 371 Date: Apr. 9, 2008

(30) Foreign Application Priority Data
Feb. 19, 2004 (FR) 0401684

(51) Int. Cl. A61B 17/08

(52) U.S. Cl. 606/157

(57) ABSTRACT

A surgical band comprising a flexible strip (2) which is designed to be closed essentially at the two ends thereof in order to form a closed loop, the strip (2) comprising an annular compression chamber (7) having a volume that can be adjusted with the injection or removal of fluid. The aforementioned chamber (7) is defined by (i) an inner wall (8A) which is formed by a membrane and (ii) a dorsal wall (8B). The dorsal wall (8B) is formed by a bead comprising an inner face which is located opposite the chamber (7) and which is provided with at least one longitudinal slot. A surgical band which is intended to be implanted in the body of a patient around one or more biological organs such as to form a pouch or conduit which can alter the flow cross-section of the organ when clamped by the band.

### TECHNICAL FIELD

This invention relates to the general technical field of surgical implants designed to be implanted in the body of a patient around a biological organ or organs, consisting of a pouch or tube, and more particularly to gastric bands designed for treating obesity by implanting a flexible gastric band, intended to form a closed loop around the stomach in order to reduce the diameter of the stoma.

This invention relates to a gastric band designed to be implanted in the body of a patient around a biological organ or organs, consisting of a pouch or a tube for altering the flow area of said organ when it is clamped by the band, said band consisting of a flexible strip designed to be substantially closed at its two ends in order to form a closed loop, said strip comprising an annular compression chamber designed to contain a filling fluid, said chamber being defined, on the one hand, by an internal wall designed to be in contact with the organ being clamped and, on the other hand, a dorsal wall.

More particularly, this invention relates to a gastroplasty band, but it also concerns a band designed to be used in treating urinary or fecal incontinence (artificial sphincter), or else a band designed to regulate the blood flow in blood vessels, this list in no way being limited.

This invention also relates to a method of manufacturing a surgical band designed to be implanted in the body of a patient around a biological organ or organs, consisting of a pouch or a tube for altering the flow area of said organ when it is clamped by the band, wherein an annular compression chamber is created, designed to contain a filling fluid, said chamber being defined, on the one hand, by an internal wall designed to be in contact with the organ being clamped and, on the other hand, a dorsal wall.

### PRIOR ART

It is already known to intervene surgically in the case of patients afflicted with extremely severe obesity (morbid obesity), i.e., in the case of patients whose weight, for example, exceeds the ideal weight by at least 50 Kg, by implanting gastroplasty bands in such patients. Such procedures make it possible to not only prevent a series of serious health problems stemming from such excessive weight, but also and above all to prevent the certain and imminent death of these patients.

As a matter of fact, it is acknowledged that patients suffering from morbid obesity see their life expectancy reduced significantly, and by at least some ten to twelve years, while at the same time suffering considerable psychological problems.

Furthermore, a whole series of related health phenomena are involved, having an impact on the appearance of related diseases, such as cardiovascular diseases, hypertension, diabetes, or even severe arthritis, in particular.

It is likewise acknowledged that, for such patients, treatments based on severe diets combined with a series of physical exercises also associated with behavior modification, in particular eating behavior, are generally unsuitable, even if these treatment methods are recognized as being the healthiest ones.

This is the reason why the effective and long-term treatments for morbid obesity involve a surgical treatment.

Generally, a distinction is made between surgical treatment techniques involving a food absorption defect, i.e., a shortening of the passage for food and digestive juices, and techniques involving a gastric restriction procedure reducing the size of the stomach.

The surgical techniques involving an absorption defect are those involving, for example, a “by-pass” technique or technique for diverting the small intestine, or else those using a separation of the passage of food relative to the digestive juices.

These techniques are now rarely used, because they can cause severe complications for the patient and, in every instance, require a major surgical procedure.

This is the reason why there is now a tendency to favor the surgical techniques that use gastric restriction to reduce food intake.

These widely known techniques involve the use of gastroplasty bands implanted around the stomach of the patient, with a view to reducing its size as well as the diameter of its passage (stoma).

The overall structure of the gastroplasty bands used is well known and involves a flexible strip, made of an elastomer material designed to be closed at its two ends, around the stomach of the patient, by closing means, in order to reduce the diameter of the stoma opening. The closing means are generally situated on the external or dorsal portion of the flexible strip, and involve various types of locking, e.g., mechanical locking with or without suturing.

The known bands also comprise a strip with an annular compression chamber the volume or diametral expansion of which is adjustable, said chamber being capable of being connected, by means of a catheter, to a device for adjusting the diameter of the chamber by injecting or withdrawing fluid. Thanks to this special feature, it is thereby possible, using a band of fixed size or diameter, to make fine adjustments in the inside diameter of the band, by injecting or withdrawing fluid, which causes a corresponding diametral expansion or retraction of the band.

The known devices of the type mentioned above generally give satisfaction, but suffer a certain number of problems, and, in particular, patient tolerance problems.

As a matter of fact, it is proven to be of particular importance to reduce as much as possible the sensation of discomfort caused by such bands in the area where the stomach is restricted, and to prevent or reduce the appearance of tissue injuries in the restriction area.

To this end, it is necessary that the surface of the band designed to be in contact with the biological tissues of the stomach be made of a material that is particularly soft to the touch, and that this surface also have a smooth and even texture and, in particular, be devoid of folds.

In particular, in order to comply with this requirement of being atraumatic, without also sacrificing the sturdiness of the band, the applicant has proposed a gastroplasty band equipped with a dorsal reinforcement made of a first elastomer material whose hardness is greater than that of a second elastomer material of which the lateral walls of the annular compression chamber are made, and in particular the walls designed to come into contact with the stomach tissues.

Even though it actually proves to offer an excellent compromise between atraumatism, sturdiness and simplicity of design, it remains a fact that this band is relatively complicated to manufacture, since it implements an operation for over-molding separate elements made of different materials.

Furthermore, even though it represents an important step towards eliminating folds around the area of contact with the stomach, it is proven that the design for this band does not necessarily completely eliminate the probability of folds or discontinuities occurring in this area of contact.

### DISCLOSURE OF THE INVENTION

Consequently, the object assigned to the invention aims to propose a novel surgical band, in particular a gastric band, that makes it possible to remedy the various disadvantages listed above and that is particularly smooth and atraumatic, so as to be well-tolerated by the patient, while at the same time being sturdy, simple in design and low in cost.

Another object of the invention aims to propose a novel surgical band, in particular a gastric band, whose atraumaticity is particularly optimized.

Another object of the invention aims to propose a novel surgical band, in particular a gastric band, which enables fine adjustments in the cross-section of the stoma opening.

Another object of the invention aims to propose a novel surgical band, in particular a gastric band, whose manufacture uses a minimum of different components.

Another object of the invention aims to propose a novel surgical band, in particular a gastric band, which is extremely simple to manufacture, while at the same time being particularly compact and lightweight.

Another object of the invention aims to propose a novel surgical band, in particular a gastric band, which offers an excellent compromise between atraumatism and simplicity of design and manufacture.

The object assigned to the invention also aims to propose a novel method of manufacturing a surgical band, in particular a gastric band, said method being particularly simple and fast, while at the same time making it possible to obtain a sturdy and atraumatic surgical band.

Another object of the invention aims to propose a novel method of manufacturing a surgical band, in particular a gastric band, which is particularly economical and easy to use.

Another object of the invention aims to propose a novel method of manufacturing a surgical band, in particular a gastric band, making it possible to obtain a band whose atraumaticity is particularly optimized.

Another object of the invention aims to propose a novel method of manufacturing a surgical band, in particular a gastric band, which makes it possible to obtain a band enabling fine adjustments in the cross-section of the stoma opening.

Another object of the invention aims to propose a novel method of manufacturing a surgical band, in particular a gastric band, making it possible to reduce the number of manufacturing steps.

The objects assigned to the invention are achieved with the aid of a surgical band designed to be implanted in the body of a patient around a biological organ or organs, consisting of a pouch or a tube for altering the flow area of said organ when it is clamped by the band, said band consisting of a flexible strip designed to be substantially closed at its two ends in order to form a closed loop, said strip comprising an annular compression chamber designed to contain a filling fluid, said chamber being defined, on the one hand, by an internal wall designed to be in contact with the organ being clamped and, on the other hand, a dorsal wall, characterized in that said dorsal wall consists of a bead having an inner face situated opposite the chamber, said inner face being provided with at least one longitudinal slot for influencing the deformation of the internal wall with a view to limiting the presence of surface irregularities in the area of the internal wall, when the strip forms a closed loop.

The objects assigned to the invention are also achieved with the aid of a method of manufacturing a surgical band designed to be implanted in the body of a patient around a biological organ or organs, consisting of a pouch or a tube for altering the flow area of said organ when it is clamped by the band, said band consisting of a flexible strip designed to be substantially closed at its two ends in order to form a closed loop, said strip comprising an annular compression chamber designed to contain a filling fluid, said chamber being defined, on the one hand, by an internal wall designed to be in contact with the organ being clamped and, on the other hand, a dorsal wall, characterized in that said dorsal wall consists of a bead having an inner face situated opposite the chamber, said inner face being provided with at least one longitudinal slot for influencing the deformation of the internal wall with a view to limiting the presence of surface irregularities in the area of the internal wall, when the strip forms a closed loop.

### SUMMARY DESCRIPTION OF THE DRAWINGS

Other objects and advantages of the invention will become more apparent upon reading the following description, as well as with the aid of the appended drawings, given for purely non-limiting and illustrative purposes, in which:

FIG. 1 is a lateral perspective view showing a gastroplasty band in accordance with the invention, in its unclamped position.

FIG. 2 is a view identical to that of FIG. 1, showing a gastroplasty band in accordance with the invention, in its closed position.

FIG. 3 is a cross-sectional view showing a first alternative embodiment of a gastroplasty band in accordance with the invention.

FIG. 4 is a cross-sectional view showing a second alternative embodiment of a gastroplasty band in accordance with the invention.

### BEST MODE OF CARRYING OUT THE INVENTION

In the following description, for illustrative purposes only and for the sake of descriptive simplicity, reference will be made to a gastroplasty band (or gastric band) designed to be implanted around the stomach in order to reduce the diameter of the stoma opening, or designed to be implanted around the oesophagus.

However, the invention is in no way limited to this application and, on the contrary, aims to also cover other surgical bands, and in general surgical bands designed to be implanted in the body of a patient around a biological organ or organs, consisting of a pouch or a tube for altering the flow area of said organ, when it is clamped by the band.

As an example, bands used to treat urinary or fecal incontinence may be cited, or else those used around blood vessels for regulating the blood flow. In the case of treating urinary incontinence, the band will be implanted around the bladder or urinary canal, and in the case of treating fecal incontinence, it will be implanted around the gastro-intestinal tract, and in particular around the anal structures of the intestine.

FIGS. 1 to 4 show a gastroplasty band 1 in accordance with the invention, consisting of a flexible strip 2 preferably made of a elastomer material, e.g., silicone, designed to be substantially closed at its two ends 3, 4 around the stomach of a patient, by means of closing means 5, 6, in order to form a closed loop, with a view to reducing the diameter of the stoma opening.

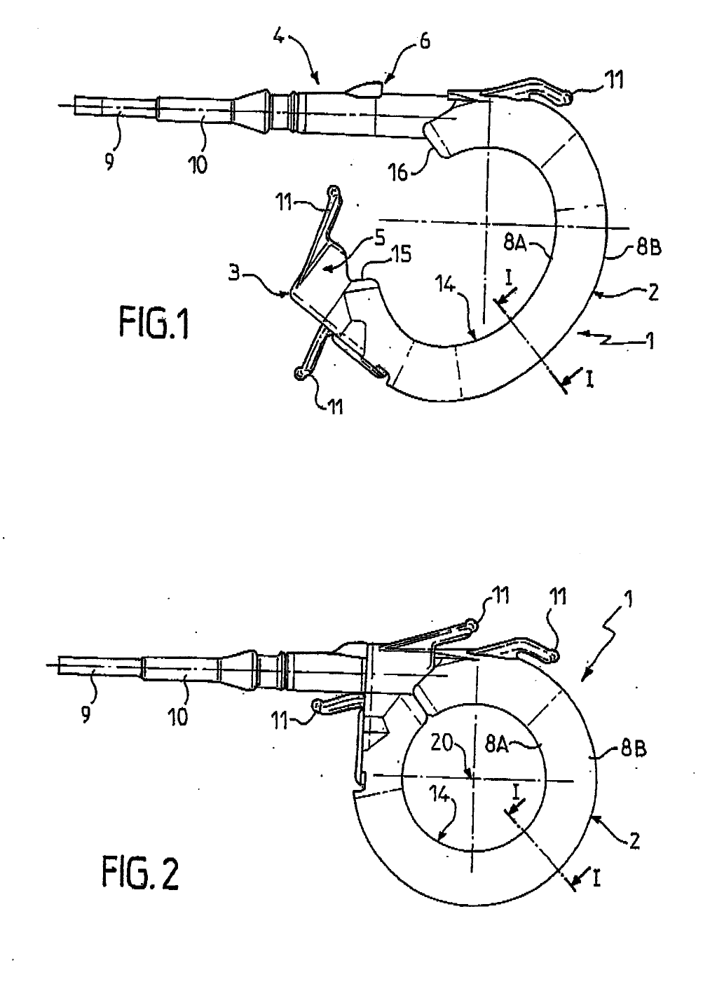

The closed position of the band is shown in FIG. 2, a position in which the closing means 5, 6 cooperate with each other to ensure that the band 1 is locked.

Preferentially, the surgical band in accordance with the invention has a substantially circular memory shape, so as to facilitate positioning of the band by the surgeon, since in its open, loose resting position (FIG. 1) the band already has an almost circular or substantially circular shape close to its final configuration shown in FIG. 2.

Advantageously, the gastroplasty band 1 in accordance with the invention may be provided with one or more gripping tabs 11 arranged at predetermined locations, e.g., near the ends 3, 4, so as to facilitate handling of the band, and in particular its closure, and above all its opening or unlocking.

According to the invention, the flexible strip 2 comprises a compression chamber 7 running longitudinally over the major portion of the length of the flexible strip 2. The closing means 5, 6 being integral with the ends 3, 4 of the flexible strip 2 and extending outwardly from said strip 2, the compression chamber 7 advantageously terminates in two substantially planar cross sections 15, 16, in order to come to bear against one another in the closed position of the band (FIG. 2), so as to form an annular compression chamber 7 capable of clamping around the stomach over an angular area equal to or substantially equal to 360 degrees.

The annular compression chamber 7 is designed to contain a filling fluid, in order to give the band its functional dimensions enabling it to reduce the flow area of the stomach to a predetermined dimension, when it surrounds an organ such as the stomach, based on the amount of fluid present in the chamber. In other words, the chamber 7 is designed to be “inflated” by the filling fluid so that the band has a contact surface with the stomach (or another organ in the case of an application other than gastroplasty) that is relatively flexible and of a predetermined diameter.

Preferentially, and in a manner known per se, the annular compression chamber 7 has an adjustable volume, i.e., its diametral expansion can be adjusted to expand or retract, so as to concomitantly adjust the diameter of the stoma opening. More particularly, the chamber 7 is a chamber having a volume that can be adjusted by injecting or withdrawing filling fluid. To this end, the annular compression chamber 7 is preferably connected, via the opening 8 and via a catheter 9 associated with the opening 8, to a device (not shown in the figures) for adjusting the diameter of said chamber 7 by injecting or withdrawing fluid. In a known manner, the adjusting device consists of a miniaturized housing that can be implanted beneath the skin of the patient, the housing comprising a self-sealing membrane designed to be pierced with a syringe making it possible to inject or withdraw a certain amount of fluid (generally saline) serving to ensure the variation in volume of the annular compression chamber 7. As illustrated in FIGS. 1 and 2, the catheter 9 can be connected to the flexible strip 2 by means of a connecting member, such as an end fitting 10.

Without necessarily exceeding the scope of the invention, it is entirely foreseeable, of course, for the chamber 7 to have a fixed and non-adjustable predetermined volume, obtained, for example, during manufacture of the band, by means of a single and definitive filling of the chamber 7 with a predetermined amount of filling fluid, said chamber 7 then being permanently sealed, so that any fluid-injecting or withdrawing operation is prohibited during use of the band.

According to the invention, the chamber 7 is longitudinally defined, on the one hand, by an internal wall 8A and, on the other hand, by an opposing dorsal wall 8B.

The internal wall 8A is advantageously formed by a membrane, i.e., a thin and flexible partition, preferably solid, having an elastic property.

The internal wall 8A is advantageously designed to come into contact, or to be in contact with the biological organ to be clamped, in this case the stomach. In this regard, the internal wall 8A is preferably situated on the inside perimeter 14 of the flexible strip 2.

The internal wall 8A is made of a first elastomer material, of the silicone type, which preferably has a smooth and biocompatible property, so as to be easily tolerated by the stomach tissues. Preferentially, this first elastomer material has a hardness ranging between 25 and 45 Shore A, and even more preferentially a hardness on the order of 30 Shore A.

According to one important characteristic of the invention, the dorsal wall 8B consists of a bead, i.e., an elongated strip, preferably solid and heavy, the cross section of which, contrary to that of a membrane, runs in two directions in space in a substantially equivalent or at least comparable manner.

In other words, the dorsal wall 8B has an average thickness that is significantly greater than that of the internal wall 8A, and, for example, at its maximum, possibly reaching 5 to 10 times the thickness of said internal wall 8A.

This bead forming dorsal wall 8B is positioned on the outside of the flexible strip 2, i.e., on the outside perimeter of the band when the latter occupies its closed position, as shown in FIG. 2.

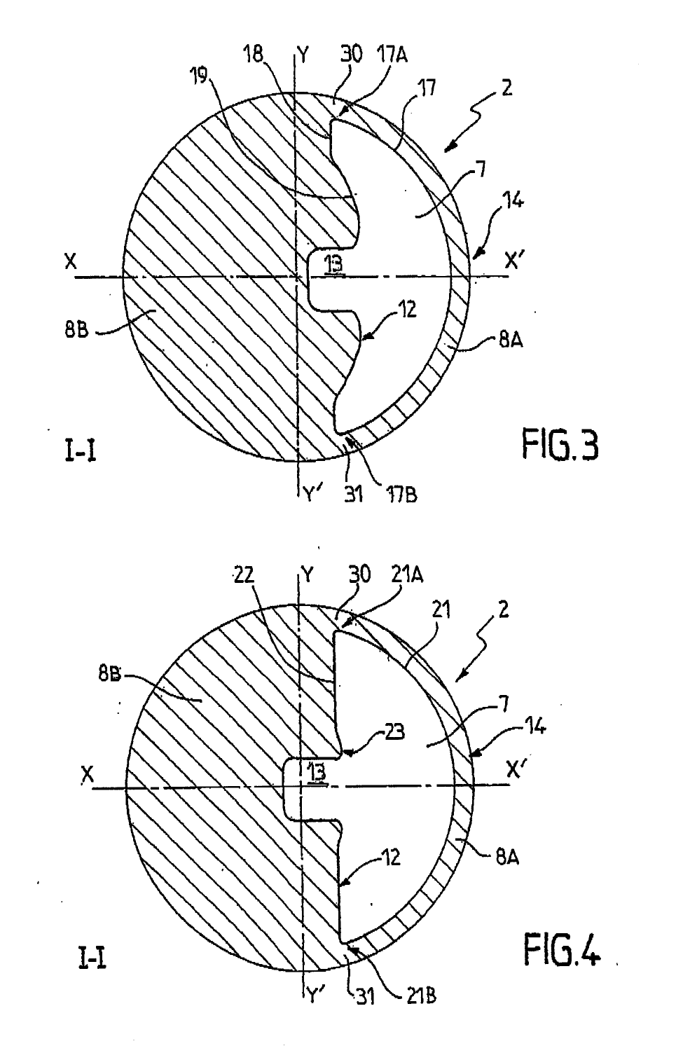

As shown in FIGS. 3 and 4, the internal 8A and dorsal 8B walls are connected at an upper junction point 30 and a lower junction point 31, so as to define the chamber 7 laterally.

The bead forming dorsal wall 8B also has an inside face 12 situated opposite the chamber 7, i.e., forming the interface between the dorsal wall 8B and the chamber 7.

Said inside face 12 is provided with at least one longitudinal slot 13 influencing the deformation of the internal wall 8A with a view to limiting the presence of surface irregularities in the area of the internal wall 8A, when the strip 2 forms a closed loop.

In other words, a slot or notch 13 is arranged on the inside face 12, said slot 13 preferably running substantially along the entire length of the bead forming the dorsal wall 8B, like a groove.

The longitudinal slot 13 opens out into the chamber 7, i.e., it forms a groove on the surface of the inside face 12, said groove thereby forming a hollow in the face 12, a hollow that communicates directly with the chamber 7.

As already stated previously, the longitudinal slot (or slots) 13 is made in the inside face 12 in order to produce a modification in the mechanical behavior of the internal wall 8A, which, in particular, results in preventing said internal wall 8A from folding, when the flexible strip 2 is closed.

In particular, in the case of an adjustable-volume chamber 7, it has been observed that the presence of one or more slots 13 made it possible to “accommodate” the variations in surface area of the internal wall 8A brought about by the variations in volume of the chamber 7, and to thereby substantially prevent the formation of folds or irregularities that might appear as a result of these variations in surface area.

Hereinafter, reference will be made to a bead whose inside face 12 is provided with a single and only longitudinal slot 13, it being understood that, without necessarily exceeding the scope of the invention, it is entirely foreseeable for the inside face 12 to be provided with several slots, possibly having different profiles, directions and dimensions.

Based on the teaching of the invention, those skilled in the art will be able to position, shape and size the slot (or slots) 13 in order to obtain and optimize the desired “anti-folding” effect of the internal wall 8A, which will thereby continuously retain a substantially smooth property.

Thus, the overall principle of the invention is based on the association of a deformable wall, designed to come into contact with the stomach, and a more rigid bead provided with at least one slot, said slotted bead and membrane defining a chamber designed to accommodate a inflating fluid, the addition of said inflating fluid causing the membrane to deform in a centripetal direction.

It has been observed by the applicant that the association of the aforesaid components resulted in the obtainment of a flexible strip 2 the compression of which is primarily if not exclusively centripetal, i.e., directed substantially towards the theoretical center 20 of the band, while at the same time having, in the area of the inside diameter 14 of the band 1 designed to come into contact with the stomach, a particularly smooth and even surface texture, substantially devoid of any fold, irregularity or discontinuity.

Therefore, the invention makes it possible to obtain an internal area 14 of the band that is substantially smooth and without folds, regardless of the level of filling (and thus compression) of the chamber 7, this effect being obtained thanks to the presence of the slot 13. In passing, it may be noted that a function of increasing the moment of inertia of the cross section of the bead 8B might perhaps be associated with the slot 13.

Advantageously, the bead forming the dorsal wall 8B has a homogeneous property, i.e., in particular, it is devoid of any irregularities in material or properties. Preferably, the longitudinal slot 13 is made entirely within the bead and, in this regard, is defined exclusively by the homogeneous material forming the bead.

Advantageously, the dorsal wall 8B is made of a second elastomer material, e.g., silicone.

Preferably, said second material is identical, i.e., similar in every respect (nature, composition, properties) to the first material of which the internal wall 8A is made.

Preferably, said first and second materials have substantially identical, or at least comparable, hardness levels.

Therefore, contrary to the devices of the prior art, the invention may make it possible to obtain an excellent technical atraumatic effect, greater than that obtained in the prior art, while at the same time using a single and only elastomer material to produce the chamber, instead of two materials having different hardness levels, as in the prior art.

Of course, that is of considerable interest from an industrial and economic viewpoint, since the use of a single material serves to facilitate the manufacturing operations and thus reduce the cost of the band.

However, it is entirely foreseeable for the dorsal 8B and internal 8A walls to be made of different materials, without exceeding the scope of the invention.

Advantageously, the internal wall 8A is made integral with the dorsal wall 8B, i.e., from their origin, at the moment they come into being, the internal 8A and dorsal 8B walls form a one-piece and homogeneous unitary assembly, without there having been any need for an operation to assemble them together (gluing, over-molding).

In this way, a particularly compact and sturdy flexible strip 2 is obtained directly, at a lower cost.

Advantageously, the inside face 12 of the bead forming the dorsal wall 8B is provided with a single longitudinal slot 13 positioned substantially at the center of said face 12, as shown in FIGS. 3 and 4.

Preferentially, the longitudinal slot 13 has a rectangular-shaped cross section (U-shaped slot), as shown in FIGS. 3 and 4. However, it is entirely foreseeable for the longitudinal slot to assume another cross-sectional profile, e.g., triangular (V-shaped slot), without necessarily exceeding the scope of the invention.

The two embodiments shown respectively in FIGS. 3 and 4 will now be described more specifically.

In these two embodiments, the flexible strip 2 appears in the form of a solid tube having a cross section whose contour is substantially elliptical. The small axis of symmetry X-X′ of said ellipse runs in a substantially radial direction, defined by the closed band 1 with center 20, while the large axis Y-Y′ runs, in a conventional manner, perpendicular to the small axis X-X′.

This elliptical shape makes it possible to give the strip 2 a relatively wide bearing surface 14, certainly greater than that of conventional annular chambers having a circular cross section. This relatively wide or, in any event, increased bearing surface makes it possible to reduce the contact pressure between the stomach and the ring, which further reduces injury to the stomach tissues.

Advantageously, the elliptical cross section of the strip 2 will remain substantially constant over the entire developed length of said strip 2.

The solid tube forming the strip 2 is hollowed out longitudinally so as to form both the chamber 7 and the longitudinal slot 13. Said chamber 7 and slot 13 communicate to form a single cavity 7, 13 that has a cross-sectional shape substantially resembling that of a mushroom whose stem is formed by the slot 13 while the cap is formed by the chamber 7.

The first alternative embodiment shown in FIG. 3 will now be described in greater detail.

In this alternative, the cross section of the chamber 7, which forms the previously mentioned mushroom cap, has an overall substantially crescent-like appearance, i.e., like a crescent moon (or disk), said cross section of the chamber 7 is defined by two curved lines, curved in the same direction and joined at their ends.

Thus, the cross section of the chamber 7 shown in FIG. 3 is defined by a first curved line 17, which can be equated substantially to an ellipse portion, an ellipse whose small axis of symmetry is coincident with the small axis X-X′ of the ellipse forming the contour of the flexible strip 2.

The first curved line 17 runs between a first end 17A and a second end 17B.

This first curved line 17 is connected at its first end 17A to a first rectilinear section 18, which runs substantially parallel to the large axis Y-Y′ of the ellipse forming the contour of the flexible strip 2. This first rectilinear section is itself extended by a second curved line 19, curved towards the inside of the chamber 7, and which runs substantially at a tangent to the rim of the slot 13.

Similar arrangements are provided at the second end 17B of the first curved line 17, with the result being that the assembly formed by the chamber 7 and the slot 13 is symmetrical in relation to the small axis X-X″.

As shown in FIG. 3, the assembly formed by the chamber 7 and the slot 13 is cross-sectionally positioned between the large axis Y-Y′ and the internal wall 8A.

For illustrative purposes, the following sizing may be adopted for the various components of the ring shown in FIG. 3:
thickness of the internal wall 8A equal to 1.1 mm;
size of the ellipse forming the contour of the flexible strip 2, along the axis X-X′ equal to 9.5 mm;
size of the ellipse forming the contour of the flexible strip 2 along the axis Y-Y′ equal to 12 mm;
width of the slot 13 in the direction Y-Y′ equal to 1.2 mm;
distance between the peak of the first curved line 17 and the bottom of the slot 13 equal to 3.75 mm;
depth of the slot equal to 1.4 mm.

It is well understood that the various values indicated above in no way limit the scope of the invention, and that these values, in particular, may be adapted by those skilled in the art, within the framework of these design operations.

The alternative embodiment shown in FIG. 4 will now be described in detail.

In this alternative, the cross section of the chamber 7, which forms the previously mentioned mushroom cap, has an overall substantially quarter-like appearance, i.e., like a quarter moon (or disk), said cross section of the chamber 7 is defined by a curved line whose ends are joined by a substantially straight line.

Thus, the cross section of the chamber 7 shown in FIG. 4 is defined by a first curved segment 21, which may be substantially equated to an ellipse portion, an ellipse whose small axis of symmetry is coincident with the small axis X-X′ of the ellipse forming the contour of the flexible strip 2.

The first curved segment 21, runs between a first end 21A and a second end 21B. This first curved segment 21 is connected at its first end 21A to a rectilinear segment 22, which runs substantially parallel to the large axis Y-Y′ of the ellipse forming the contour of the flexible strip 2. This rectilinear segment 22 itself comes alongside the rim of the slot 13, via a second curved segment 23 curved towards the chamber 7, which second curved segment 23 creates a transition curve with the rim of the slot 13. The second curved segment 23 thus produces a slight protuberance near the rim of the slot 13.

Similar arrangements are provided with respect to the second end 21B of the second curved segment 21, with the result being that the assembly formed by the chamber 7 and the slot 13 is symmetrical in relation to the small axis X-X′.

As shown in FIG. 4, the slot 13 may be arranged on either side of the large axis Y-Y′, in the direction X-X′.

For illustrative purposes, the following sizing may be adopted for the various components of the ring shown in FIG. 4:
thickness of the internal wall 8A equal to 0.6 mm;
size of the ellipse forming the contour of the flexible strip 2, along the axis X-X′ equal to 11.5 mm
size of the ellipse forming the contour of the flexible strip 2 along the axis Y-Y′ equal to 12 mm;
width of the slot 13 in the direction Y-Y′ equal to 2.2 mm;
distance between the peak of the first curved segment 21 and the bottom of the slot 13 equal to 6.35 mm;
depth of the slot equal to 2 mm.

It is well understood that the various values indicated above in no way limit the scope of the invention, and that these values, in particular, may be adapted by those skilled in the art, within the framework of these design operations.

Preferentially, and whichever embodiment is adopted, the cross section of the slot 13, just like that of the chamber 7, will be substantially constant over the entire developed length of the strip 2.

The invention also relates to a method of manufacturing a surgical band 1 in accordance with the invention, and in particular a gastroplasty band designed to be implanted around the stomach or oesophagus.

According to the invention, during implementation of this method, an annular compression chamber 7 is created, which is designed to contain a filling fluid, said chamber preferably being a chamber having a volume that can be adjusted by injecting or withdrawing filling fluid, said chamber 7 being defined, on the one hand, by an internal wall 8A, preferably consisting of a membrane, and, on the other hand, a dorsal wall 8B.

The manufacturing method in accordance with the invention includes a step for producing a bead designed to form the dorsal wall 8B, said bead having an inside face 12 designed to be located opposite the chamber 7.

According to another important characteristic of the method in accordance with the invention, the latter also includes a step for producing at least one longitudinal slot 13 on said inside face 12 of the bead, in order to influence the deformation of the internal wall 8A with a view to limiting the presence of surface irregularities on the internal wall 8A, when the strip 2 forms a closed loop. The function of the slot 13 has already been described in the preceding, and it is not necessary to describe it here again in detail.

Advantageously, the chamber 7, the internal 8A and dorsal 8B walls, as well as said at least one slot 13, are produced by a single operation of injecting an elastomer material into a mold provided with at least one cavity itself comprising at least one core.

Thus, the method in accordance with the invention advantageously makes it possible to obtain the chamber 7, the internal 8A and dorsal 8B walls, as well as said at least one slot 13, simultaneously and in a single step.

Even more preferentially, said single injection operation is carried out with a single elastomer material, such as biomedical-grade silicone, i.e., this injection operation makes it possible to obtain the flexible strip 2 in a single step.

However, it is entirely foreseeable for the internal 8A and dorsal 8B walls to be obtained following two separate injection operations, involving different materials, and for said internal 8A and dorsal 8B walls to then be assembled together at their junction points 30, 31 by any conventional method of the gluing type, over-molding type or the like.

The method as described above thus makes it possible to obtain, very simply and in a relatively brief period of time, a compact, homogenous and one-piece chamber 7.

It is further interesting to note that, by reason of its simplicity, this method can be easily automated, while involving a minimum number of manual operations.

Finally, the invention also relates to a medical treatment for morbid obesity including the steps of installing, controlling and adjusting a gastric band, and particularly the diameter thereof, in accordance with the invention.

### POSSIBILITY OF INDUSTRIAL APPLICATION

The invention finds its industrial application in the design and manufacture of surgical bands designed to be implanted around a biological organ or organs, consisting of a pouch or a tube for altering the flow area of said organ when it is clamped by the band, and in particular gastric bands designed for treating obesity.

1. A surgical band designed to be implanted in the body of a patient around a biological organ or organs, consisting of a pouch or a tube for altering the flow area of said organ when it is clamped by the band, said band comprising:
a flexible strip designed to be substantially closed at its two ends in order to form a closed loop; said strip comprising an annular compression chamber designed to contain a filling fluid; said chamber being defined by an internal wall designed to be in contact with the organ being clamped and a dorsal wall,
herein said dorsal wall consists of a bead having an inner face situated opposite the chamber, said inner face has at least one longitudinal slot for influencing the deformation of the internal wall with a view to limiting the presence of surface irregularities in the area of the internal wall, when the strip forms a closed loop.

2. The band of claim 1, wherein the internal wall comprises a membrane.

3. The band of claim 1, wherein the chamber is a chamber having a volume that can be adjusted by injecting or withdrawing said filling fluid.

4. The band claim 1, wherein the bead has a homogeneous property and the longitudinal slot is arranged entirely within the bead.

5. The band of claim 1, wherein the internal wall comprises a first elastomer material, and the dorsal wall comprises a second elastomer material.

6. The band of claim 5, wherein said first and second materials are identical.

7. The band of claim 6, wherein said first and second materials have substantially identical hardness levels.

8. The band of claim 1, wherein the internal wall is made integral with the dorsal wall.

9. The band of claim 1, wherein the inner face of the bead has a single longitudinal slot positioned substantially at the center of said face.

10. The band claim 1, wherein the longitudinal slot has a substantially rectangular-shaped cross section.

11. The band of claim 1, wherein the strip is in the form of a solid tube having a substantially elliptical cross section, said tube being hollowed out so as to form both the chamber and the longitudinal slot, said chamber and slot communicating in order to form a single cavity whose cross-sectional shape substantially resembles that of a mushroom whose stem is formed by the slot, while the cap is formed by the chamber.

12. The band of claim 11, wherein the cross section of the chamber has an overall quarter-like appearance.

13. The band of claim 11, wherein the cross section of the chamber has an overall crescent-like appearance.

14. The band of claim 1, wherein the band constitutes a gastroplasty band designed to be implanted around the stomach or the oesophagus.

15. A method of manufacturing a surgical band designed to be implanted in the body of a patient around a biological organ or organs, the band comprising a pouch or a tube for altering the flow area of said organ when the organ is clamped by the band in which is made an annular compression chamber, designed to contain a filling fluid;
said chamber being defined by an internal wall designed to be in contact with the organ being clamped and a dorsal wall,
said method comprising
(a) making the bead intended to form the dorsal wall; said bead having an inner face situated opposite the chamber, and
(b) creating said interior surface, with at least one longitudinal slot for influencing the deformation of the internal wall with a view to limiting the presence of surface irregularities in the area of the internal wall, when the strip forms a closed loop.

16. The Method of claim 15, wherein the internal wall comprises a membrane.

17. The method of claim 15, wherein the chamber is a chamber having a volume that can be adjusted by injecting or withdrawing filling fluid.

18. The method of claim 15, wherein the chamber, the internal and dorsal walls, as well as said at least one slot are produced by a single operation of injecting a single elastomer material into a mold.

19. The method of claim 15, wherein the method is used to manufacture a gastroplasty band designed to be implanted around the stomach or the oesophagus.

20. The band of claim 2, wherein the chamber is a chamber having a volume that can be adjusted by injecting or withdrawing said filling fluid.

21. The method of claim 16, wherein the chamber is a chamber having a volume that can be adjusted by injecting or withdrawing filling fluid.
